(19) United States
(12) Patent Application Publication
(10) Pub. No.: US 2002/0018766 A1
(43) Pub. Date: Feb. 14, 2002

(54) Genes differentially expressed in cancer cells to design cancer vaccines

(76) Inventors: Bruce L. Roberts, Southboro, MA (US); Srinivas Shankara, Shrewsbury, MA (US); Charles A. Nicolette, Framingham, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/826,609

(22) Filed: Apr. 5, 2001

Related U.S. Application Data
(63) Continuation of application No. PCT/US99/23166, filed on Oct. 4, 1999.
(60) Provisional application No. 60/103,220, filed on Oct. 5, 1998.

(51) Int. Cl. A61K 48/00; C12Q 1/68; A61K 39/395; A61K 38/19

(52) U.S. Cl. 424/93.21; 424/85.1; 424/155.1; 435/6

(57) ABSTRACT

The present invention calls utilized genes differentially expressed in target cells to design vaccines to generate an immune response. Unlike prior art methods that seek to identify antigenic proteins from phenotypic analysis, the subject method applies functional genomics for antigen identification. The method is exemplified herein and therefore provides compositions and methods for inducing an immune response against gp 100 melanoma cells and for inducing an immune response against HER-2⁺cells. Cancer vaccines and adoptive immunotherapeutic methods to treat and prevent conditions associated with the presence of these cells in a subject also are provided. The methods can be practiced by administering the appropriate gene or cancer vaccine, antibody, protein, polypeptide, antigen-presenting cell or immune effector cell.

### CROSS-REFERENCE TO RELATED APPLICATIONS

This application claims the benefit under 35 U.S.C. §119(e) of U.S. Provisional Application No. 60/103,220, filed, Oct. 5, 1998, the contents of which are hereby incorporated by reference into the present disclosure.

### TECHNICAL FIELD

This invention is in the fields of molecular biology, cell biology and immunology. More particularly, the invention uses techniques of functional genomics to identify antigenic proteins and polypeptides.

### BACKGROUND OF THE INVENTION

Investigators have sought to elicit antigen specific T cell responses in the hopes of creating an anti-tumor cell immune response that might lead to the eradication of tumor cells. To date, 4 classes of tumor antigens have been identified: differentiation antigens which are self proteins over-expressed by tumor cells; viral antigens such as BPV16E6 and E7; the cancer/testes family of antigens typified by MAGE; and mutated proteins such as ras or p53. Of the differentiation antigens, the vast majority are melanoma associated antigens and attempts to identify self antigens over-expressed by lung, prostate, breast or colon carcinomas that might be good candidates as targets for cytotoxic T cells have largely been unsuccessful. Thus the vast majority of cancer immunotherapy trials conducted to date have been for the treatment of melanoma and little by way of immunotherapy is available to offer patients suffering with other malignant diseases. The present invention addresses the limitation of a scarcity of tumor antigens that have been identified for malignancies other than melanoma and other pathologies as well.

### DISCLOSURE OF TEE INVENTION

The present invention uses differentially expressed genes in target cells to design vaccines.

This invention provides a method for identifying putative antigens by comparing the expression level of transcripts isolated from a target cell with a control cell, and identifying the transcripts overexpressed or exclusively expressed in the target cell as compared to the control cell. The sequence of the cDNA corresponding to the tag is isolated and its protein product identified. If the protein is immunogeneic, it is useful as a cancer vaccine or in adoptive immunotherapy. Unlike prior art methods that seek to identify antigenic proteins from phenotypic analysis, the subject method applies functional genomics for antigen identification.

This invention also provides a method for inducing an immune response against a target cell in a subject by delivering to the subject an effective amount of an antigenic peptide that is uniquely expressed or overexpressed in the target cell and has not been previously identified as having the ability to induce an immune response in the subject, whereby an immune response is mounted against the target cell.

The method is exemplified herein and therefore provides compositions and methods for inducing an immune response against gp100 melanoma cells. In a further embodiment, compositions and methods for inducing an immune response against HER-2⁺ cells are provided herein. Cancer vaccines and adoptive immunotherapeutic methods to treat and prevent conditions associated with the presence of these cells in a subject also further provided. The methods can be practiced by administering the appropriate gene or cancer vaccine, antibody, protein, polypeptide, antigen-presenting cell or immune effector cell.

### BRIEF DESCRIPTION OF THE FIGURE

FIGS. 1A and 1B graphically show the relative susceptibility of the cell lines to lysis by a gp100 specific cytotoxic T lymphocyte.

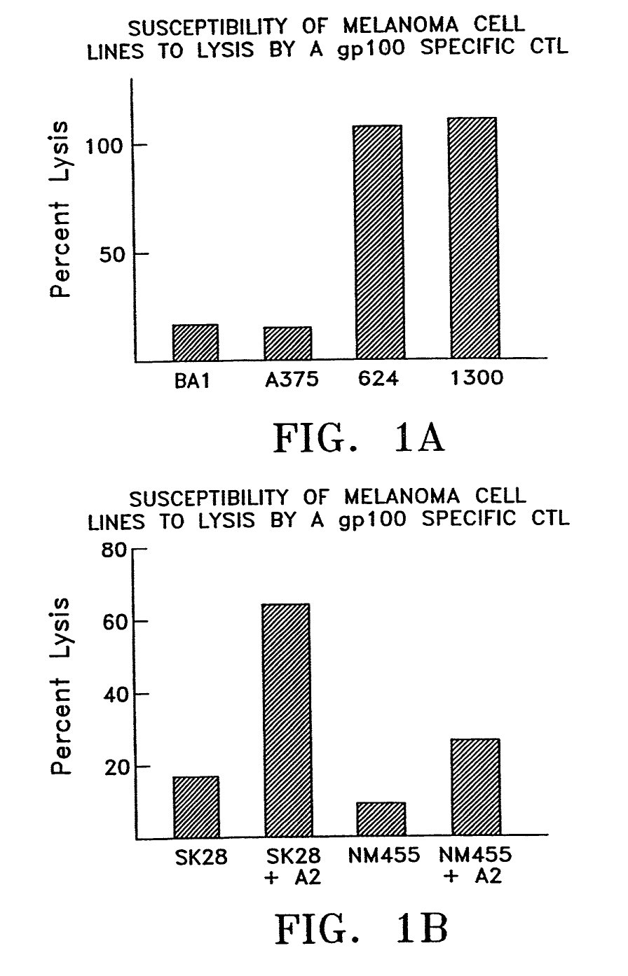

### MODES FOR CARRYING OUT THE INVENTION

Various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention will employ, unless otherwise indicated, conventional techniques of molecular biology, microbiology, cell biology and recombinant DNA, which are within the skill of the art. See, e.g. Sambrook, et al. MOLECULAR CLONING: A LABORATORY MANUAL, 2^{nd} edition (1989); CURRENT PROTOCOLS IN MOLECULAR BIOLOGY (F. M. Ausubel et al. eds.(1987)); the series METHODS IN ENZYMOLOGY (Academic Press, Inc.); PCR2: A PRACTICAL APPROACH (M. J. MacPherson, B. D. Hames and G. R. Taylor eds. (1995)); and ANIMAL CELL CULTURE (R. I. Freshney, ed. (1987)).

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein a second polynucleotide “corresponds to” another (a first) polynucleotide if it is related to the first polynucleotide by any of the following relationships:

1) The second polynucleotide comprises the first polynucleotide and the second polynucleotide encodes a gene product.

2) The second polynucleotide is 5′ or 3′ to the first polynucleotide in cDNA, RNA, genomic DNA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide, for example a promoter sequence. The first and second polynucleotide may be fragment of a gene coding for a gene product. The second polynucleotide may be an exon of the gene while the first polynucleotide may be an intron of the gene.

3) The second polynucleotide is the complement of the first polynucleotide.

The “genotype” of a cell refers to the genetic makeup of the cell and/or its gene expression profile. Modulation of the genotype of a cell can be achieved by introducing additional DNA or RNA either as episomes or as an integral part of the chromosomal DNA of the recipient cell. The genotype can also be modulated by altering the expression level, e.g. mRNA abundance, of a particular gene using agents that regulate gene expression.

A “database” denotes a set of stored data which represent a collection of sequences including nucleotide and peptide sequences, which in turn represent a collection of biological reference materials.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

The term “antigen” is well understood in the art and includes substances which are immunogenic, i.e., immunogens, as well as substances which induce immunological unresponsiveness, or anergy, i.e., anergens.

A “self-antigen” also referred to herein as a native or wild-type antigen is an antigenic peptide that induces little or no immune response in the subject due to self-tolerance to the antigen. An example of a self-antigen is the human melanoma antigen gp100.

The term “tumor associated antigen” or “TAA” refers to an antigen that is associated with or specific to a tumor. Examples of known TAAs include gp100, MART and MAGE.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules. “Oligonucleotide” refers to polynucleotides of between about 5 and about 100 nucleotides of single- or double-stranded DNA. Oligonucleotides are also known as oligomers or oligos and may be isolated from genes, or chemically synthesized by methods known in the art.

The term “cDNAs” refers to complementary DNA, that is m-RNA molecules present in a cell or organism made in to cDNA with an enzyme such as reverse transcriptase. A “cDNA library” is a collection of all of the mRNA molecules present in a cell or organism, all turned into cDNA molecules with the enzyme reverse transcriptase, then inserted into “vectors”.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

A “sequence tag” or “tag” or “SAGE tag” is a short oligonucleotide containing defined nucleotide sequence that occurs in a certain position of a gene transcript. The length of a tag is generally under about 20 nucleotides, preferably between 9 to 15 nucleotides, and more preferably 10 nucleotides. The tag can be used to identify the corresponding transcript and gene from which it was transcribed. A tag can further comprise exogenous nucleotide sequences to facilitate the identification and utility of the tag. Such auxiliary sequences include, but are not limited to, restriction endonuclease cleavage sites and well known primer sequences for sequencing and cloning.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “promoter” is a region on a DNA molecule to which an RNA polymerase binds and initiates transcription. In an operon, the promoter is usually located at the operator end, adjacent but external to the operator. The nucleotide sequence of the promoter determines both the nature of the enzyme that attaches to it and the rate of RNA synthesis.

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques,

The term “aberrantly expressed” refers to nucleotide sequences in a cell or tissue which are either over-expressed or under-expressed when compared to a different cell or tissue.

A “gene delivery vehicle” is defined as any molecule that can carry inserted polynucleotides into a host cell. Examples of gene delivery vehicles are liposomes, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy and protein production and expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSLM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgibin/BLAST.

The term “immune effector cells” refers to cells capable of binding an antigen and which mediate an immune response. These cells include, but not limited to, T cells, B cells, monocytes, macrophages, NK cells and cytotoxic T lymphocytes (CTLs), for example CTL lines, CTL clones, and CTLs from tumor, inflammatory, or other infiltrates. Certain diseased tissue expresses specific antigens and CTLs specific for these antigens have been identified. For example, approximately 80% of melanomas express the antigen known as GP-100

The term “T-lymphocytes” as used herein denotes lymphocytes that are phenotypically CD3⁺, typically detected using an anti-CD3 monoclonal antibody in combination with a suitable labeling technique. The T-lymphocytes of this invention are also generally positive for CD4, CD8, or both.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin- 1 alpha (IL-lα), interleukin-11 (IL-11), MIP-lα, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

“Co-stimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells (Freeman, et al. (1993) Science 262:909-911; Young, et al. (1992) J. Clin. Invest. 90:229). Other important co-stimulatory molecules are CD40, CD54, CD80, CD86.

The terms “antigen-presenting cells” or “APCs” includes both intact, whole cells as well as other molecules which are capable of inducing the presentation of one or more antigens, preferably in association with class I MHC molecules. Examples of suitable APCs are discussed in detail below and include, but are not limited to, whole cells such as macrophages, dendritic cells, B cells, purified MHC class I molecules complexed to βP2-microglobulin; and foster antigen presenting cells.

Dendritic cells (DCs) are potent antigen-presenting cells. It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called co-stimulatory signals, is neither antigen-specific nor MFC-restricted, and can lead to a full proliferation response of T cells and induction of T cell effector functions in the presence of the first type of signals. As used herein, “dendritic cell” is to include, but not be limited to a pulsed dendritic cell, a foster cell or a dendritic cell hybrid.

A “naïve” cell is a cell that has never been exposed to an antigen.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

A “subject” is a vertebrate, preferably a mammal, more preferably a human. Mammals include, but are not limited to, murines, simians, humans, farm animals, sport animals, and pets.

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into class I and class II MHC molecllle. Class I MHC molecules include membrane heterodimeric proteins made up of an a chain encoded in the MHC associated noncovalently with β2-microglobulin. Class I MHC molecules are expressed by nearly all nucleated cells and have been shown to function in antigen presentation to CD8⁺ T. cells. Class I molecules include HLA-A, -B, and -C in humans. Class II MHC molecules also include membrane heterodimeric proteins consisting of noncovalently associated α and β chains. Class II MHC are known to participate in antigen presentation to CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a self class I or class II MHC molecule. Methods of identifying and comparing MHC are well known in the art and are described in Allen et al. (1994) Human Immun. 40:25-32; Santamaria et al. (1993) Human Immun. 37:39-50; and Hurley et al. (1997) Tissue Antigens 50:401-415.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

“Host cell” or “recipient cell” is intended to include any individual cell or cell culture that can be or has been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be procaryotic or eucaryotic, and include but are not limited to bacterial cells, yeast cells, animal cells, and mammalian cells, e.g., murine, rat, simian or human. An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody (as defined above) and its binding partner or ligand.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

An “isolated” or “enriched” population of cells is “substantially free” of cells and materials with which it is associated in nature. By “substantially free” or “substantially pure” means at least 50% of the population are the desired cell type, preferably at least 70%, more preferably at least 80%, and even more preferably at least 90%.

A “composition” is intended to mean a combination of active agent and another compound or composition, inert (for example, a detectable agent, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

This invention provides a quick and efficient method for identifying putative antigenic peptides for use in vaccines and adoptive immunotherapy. The inventors have discovered that previously characterized and uncharacterized proteins that are differentially expressed on target cells as compared to normal cells can be used in immunotherapy. The putative antigens are identified by obtaining a set of polynucleotides representing gene expression in a target cell and obtaining a set of polynucleotides representing gene expression in a control cell. The sets of polynucleotides are compared for sequence identity and expression level. The polynucleotides that are uniquely expressed or overexpressed in the target cells as compared to the normal cells are identified as putative vaccine candidates. Immunogenicity is confirmed by the ability of the protein or peptide fragment thereof being capable of raising antibodies or educating naïve immune effector cells, which in turn, lyse target cells of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹CR-release assays, or ³H-thymidine uptake assays.

“Target cells” can include, but are not limited to, neoplastic cells; drug-resistant neoplastic cells; neoplastic cells which promote angiogenesis; de-differentiated cells; differentiated cells; apoptotic cells; hyperproliferative cells; cells infected with a pathogen or drug-resistant cells infected with a pathogen. In one aspect, the target cells are those cells that have been previously identified as being particularly sensitive to lysis by T cells or reactive to an antibody.

Cancers from which cells can be obtained for use in the methods of the present invention include carcinomas, sarcomas, leukemias, and cancers derived from cells of the nervous system. These include, but are not limited to: brain tumors, such as astrocytoma, oligodendroglioma, ependymoma, medulloblastomas, and Primitive Neural Ectodermal Tumor (PNET); pancreatic tumors, such as pancreatic ductal adenocarcinomas; lung tumors, such as small and large cell adenocarcinomas, squamous cell carcinoma and bronchoalveolarcarcinoma; colon tumors, such as epithelial adenocarcinoma and liver metastases of these tumors; liver tumors, such as hepatoma and cholangiocarcinoma; breast tumors, such as ductal and lobular adenocarcinoma; gynecologic tumors, such as squamous and adenocarcinoma of the uterine cervix, and uterine and ovarian epithelial adenocarcinoma; prostate tumors, such as prostatic adenocarcinoma; bladder tumors, such as transitional, squamous cell carcinoma; tumors of the reticuloendothelial system (RES), such as B and T cell lymphoma (nodular and diffuse), plasmacytoma and acute and chronic leukemia; skin tumors, such as melanoma; and soft tissue tumors, such as soft tissue sarcoma and leiomyosarcoma.

Tumor cells are typically obtained from a cancer patient by resection, biopsy, or endoscopic sampling; the cells may be used directly, stored frozen, or maintained or expanded in culture. Samples of both the tumor and the patient's blood or blood fraction should be thoroughly tested to ensure sterility before co-culturing of the cells. Standard sterility tests are known to those of skill in the art and are not described in detail herein. The tumor cells can be cultured in vitro to generate a cell line. Conditions for reliably establishing short-term cultures and obtaining at least 10⁸ cells from a variety of tumor types is described in Dillmar, et al. (1993) J. Immun. 14:65-69. Alternatively, tumor cells can be dispersed from, for example, a biopsy sample, by standard mechanical means before use.

Tumor cells can be obtained by any method known in the art. The following is an example of one method employed by skilled artisans. Using sterile technique, solid tumors (10-30 g) excised from a patient are dissected into 5 mm³ pieces which are immersed in RPMI 1640 medium containing 0.01% hyaluronidase type V, 0.002% DNAse type I, 0.1% collagenase type IV, 50 IU/ml penicillin, 50 mg/ml streptomycin and 50 mg/ml gentamycin. This mixture is stirred for 6 to 24 hours at room temperature, after which it is filtered through a coarse wire grid to exclude undigested tissue fragments. The resultant tumor cell suspension is then centrifuged at 400×g for 10 minutes. The pellet is washed twice with Hanks balanced salt solution (HBSS) without Ca²⁺ or Mg²⁺ or phenol red, then resuspended in HBSS and passed through Ficoll-Hypaque gradients. The gradient interfaces, containing viable tumor cells, lymphocytes, and monocytes, are harvested and washed twice more with HBMSS. The harvested cells may be frozen for storage in a type-compatible human serum containing 10% (v/v) DMSO.

The terms “neoplastic cell”, “tumor cell”, or “cancer cell”, used either in the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

Using the methods described above, differentially expressed polynucleotides isolated from breast cancer cell lines were compared to normal breast cell lines. Additionally, differentially expressed polynucleotides isolated from HLA-A2 restricted gp100⁺ melanoma cell lines were compared to tags isolated from HLA-A2 restricted normal melanocytes. These analyses identified a variety of transcripts that are differentially expressed in cancer cells, some of which correspond to previously identified tumor associated antigens such as gp 100 (in melanoma) and HER-2 (in breast cancer). However, many other transcripts were identified as differentially expressed in cancer cells. The genes corresponding to these transcripts were identified. Polypeptides encoded by the genes were found to provoke immune responses (T cell or antibody mediated) against the cancer cells from which the polynucleotides were isolated. Interestingly, the peptides (cdc-related protein kinase activity and integrin alpha-3) were previously isolated and characterized, but heretofore unknown to function immunologically. The polypeptides and protein encoded thereby can be utilized to create an anti-tumor cell response that might lead to the elimination of cancer cells expressing the differentially expressed gene. This invention is not limited to the field of cancer as any differentially expressed gene or the encoded gene or the encoded protein in a target cell (whether it is malignant, benign, virally infected, abnormal or deemed dispensible) is intended to be used in a vaccine to provoke an immune response directed towards the target cell for the purpose of elimination of the target cell.

Thus, this invention provides a method for inducing an immune response against a target cell expressing a differentially expressed antigen or marker by introducing into the subject in need of such therapy an effective amount of a vaccine comprising the differentially expressed antigen or an effective amount of a cell that expresses the antigen in the context of an MHC molecule or an effective amount of immune effector cells educated against the antigen. In one embodiment, the cell is a melanoma cell that expresses cdc2-related protein kinase (hereinafter “cdc2 protein”, the sequence of which is known in the art and provided under Accession No. M65820 (www.ncbi.nlm.nih) and Ninomiya-Tsuji et al. (1991) PNAS 88:9006-9010). As used herein functionally equivalent polynucleotides and proteins can be used on the methods described herein.

The term “polypeptide having cdc2-related protein kinase activity” includes, but is not limited to polypeptides having the sequences provided in the art, analogs, allelic variants and polypeptides having conservative amino acid substitutions as compared to the cdc2 protein sequence of Ninomiya—Tsuji, et al. (1991) supra. Examples of these analogs include, but are not limited to polypeptides produced by polynucleotide sequences known in the art having cdc-2 related protein kinase activity as determined by a sequence alignment program analysis run under default prameters, but also those which hybridize under conditions of moderate or alternatively, high stringency to the sequence or those sequences that are at least 75%, or more preferably at least 80% or more preferably at least 90% or more preferably at least 95% homologous to known sequences as determined by a sequence alignment program run under default parameters.

Also within the scope of this invention are biologically active fragments of the cdc2 protein analogs, allelic variants and polypeptides having conservative amino acid substitutions. These fragments can be generated using known sequences and chemical synthesis methods or alternatively, using recombinant techniques including restriction enzyme digestion, purification and expression in a host cell. Sambrook, et al. (1989) supra.

In a separate embodiment, the cell is a breast cancer cell or a cell that expresses the HER-2 antigen and the antigen is human integrin alpha-3 chain protein (hereinafter “integrin alpha-3, the sequence of which is provided under accession number M59911 (www.ncbi.nlm.nih.gov/) and Takada et al. (1991) J. Cell Bio. 115:257-266). As used herein, the term “polypeptide having human integrin alpha-3 chain protein activity” includes, but is not limited to polypeptides having the sequences known in the art and polypeptides encoded by the polynucleotides, but also analogs, allelic variants and polypeptides having conservative amino acid substitutions as compared to the published sequences. Examples of these analogs include, but are not limited to polypeptides (produced by polynucleotide sequences) having integrin alpha-3 activity and which hybridize under conditions of moderate or alternatively, high stringency to the sequence or those sequences that are at least 75%, or more preferably at least 80% or more preferably at least 90% or more preferably at least 95% homologous to sequences known in the art as determined by a sequence alignment program run under default parameters.

Also within the scope of this invention are biologically active fragments of the integrin alpha-3 protein analogs, allelic variants and polypeptides having conservative amino acid substitutions. These fragments can be generated using the integrin alpha-3 sequences known in the art and chemical synthesis methods or alternatively, using recombinant techniques including restriction enzyme digestion, purification and expression in a host cell. Sambrook, et al. (1989) Supra.

This invention also provides methods to induce an immune response against an appropriate cell by administering to the subject an effective amount of a cancer vaccine comprising the antigen identified by the above method to the subject. As used herein, a cancer vaccine, includes but is not limited to a polynucleotide encoding the antigen or an epitopic fragment thereof or the antigen or the epitope. It also includes cells or compositions that present the antigen or epitope in a manner that activates T cells against the antigen or epitope. Examples of these include, but are not limited to antigen presenting matrices or dendritic cells that present the epitope in the context of an MHC molecule. These methods can be further modified by co-administration of an effective amount of a cytokine or a co-stimulatory molecule to the subject. This can be achieved by contacting the cell or administering to the subject an effective amount of the cytokine or co-stimulatory molecule protein, or by administering an effective amount of the gene coding for the cytokine or co-stimulatory molecule in a gene delivery vehicle or host cell.

The above methods are suitably combined with other known anti-tumor therapies or therapies yet to be discovered.

This invention further provides a method of producing a population of educated, antigen-specific immune effector cell capable of lysing a cell expressing an antigen identified by the above method by culturing naïve immune effector cells with antigen-presenting cells which express an epitope of the antigen on the surface of the cells. In one particular aspect of the invention, the antigen is cdc-2 protein kinase protein for the treatment of cells that express gp 100 antigen such as melanoma cells. In a separate embodiment, the antigen is integrin alpha-3 for the treatment of cells that express the HER-2 antigen such as breast cancer cells. The immune effector cells are administered to a subject to treat or prevent proliferation of these cells, e.g., melanoma or breast cancer cells, or to ameliorate the symptoms associated with the presence of the cells in a subject.

It is also contemplated by this invention that the immune effector cells and/or the antigen-presenting cells administered in the above methods be genetically modified to express a cytokine and/or a co-stimulatory molecule. Alternatively, compositions containing an effective amount of the cytokine and/or co-stimulatory molecule be administered with the APCs and/or immune effector cells.

This invention further provides any of the above cells or populations and a carrier, wherein the carrier includes, but is not limited to a pharmaceutically acceptable carrier or a solid support. Substantially purified and purified populations of these cells are further provided.

In one embodiment, the method further comprising contacting the cell with an effective amount of a cytokine or a co-stimulatory molecule. This can be achieved by contacting the cell with the cytokine or co-stimulatory molecule protein, or by administering a gene coding for the cytokine or co-stimulatory molecule in a gene delivery vehicle or host cell. These methods are suitably combined with other known anti-tumor therapies or therapies yet to be discovered.

The method can be practiced in vitro or in vivo. In vitro, the method provides an assay to test new anti-tumor therapies. In vitro, the method provides an assay to test new anti-tumor therapies. In vivo, the method provides a convenient animal model to test new anti-tumor therapies. When practiced in a human subject, the method is a prophylactic or therapeutic anti-tumor therapy.

This invention further provides any of the above polynucleotides, peptides, proteins, cells or populations of cells and a carrier, wherein the carrier includes, but is not limited to a pharmaceutically acceptable carrier or a solid support. Substantially purified and purified compositions are further provided herein.

This invention is not limited to embodiments wherein T cell activation is desired, but is extended to include induction of T cell anergy, e.g., autoimmune disorders, allergies, and allograft rejection.

Further provide are screens for other biological agents, proteins and small molecules that have the same function as the molecules identified above or agonists or antagonists thereof.

The following examples are intended to illustrate, but not limit the invention.

### Polynucleotide Fragments or Expression Tags

Practice of the method of this invention involves analysis of polynucleotide fragments of expressed genes. The polynucleotides are obtained from target and control cells using methods well known in the art. Many methods are known in the art to identify differentially expressed polynucleotides and each can be used to provide these polynucleotides. As used herein, the term “polynucleotide” includes SAGE tags (described below) as well as any other nucleic acid obtained from methods that yield quantitative/comparative gene expression data. Such methods include, but are not limited to cDNA subtraction, differential display and expressed sequence tag methods. Techniques based on cDNA subtraction or differential display can be quite useful for comparing gene expression differences between two cell types (described in Hedrick, et al. (1984) Nature 308:149 and Lian and Pardee (1992) Science 257:967). The expressed sequence tag (EST) approach is another valuable tool for gene discovery (described in Adams, et al. (1991) Science 252:1651), like Northern blotting, RNase protection, and reverse transcriptase-polymerase chain reaction (RT-PCR) analysis (described in Sambrook, et al. (1989) supra; Alwine, et al. (1977) PNAS 74:5350; Zinn, et al. (1983) Cell 34:865; and Veres, et al. (1987) Science 237:415). A further method utilizes differential display coupled with real time PCT and representational difference analysis (described in Lisitisyn and Wigler (1995) Meth. Enzymol. 254:291-304).

A preferred method is Serial Analysis Gene Expression or SAGE (see, U.S. Pat. No. 5,695,937) which uses sequence tags corresponding to expressed genes and was used in the Experimental Examples, below. In brief, sequence tags corresponding to the expressed gene were prepared by first obtaining cDNA from melanoma cell lines or breast cancer cell lines.

Smaller fragments of cDNA were created using a restriction endonuclease, preferably one that would be expected to cleave most transcripts at least once. Preferably, a 4-base pair recognition site enzyme was used. More than one restriction endonuclease was used, sequentially or in tandem. The cleaved cDNA was isolated by binding to a capture medium using the label attached to a primer.

The isolated defined nucleotide sequence tags were separated into two pools of cDNA. Each pool was ligated using the appropriate restriction endonucleases to linkers. The first oligonucleotide linker comprises a first sequence for hybridization of a PCR primer and the second oligonucleotide linker comprises a second sequence for hybridization of a PCR primer. In addition, the linkers further comprise a second restriction endonuclease site. The linkers were designed so that cleavage of the ligation products with the second restriction enzyme results in release of the linker having a defined nucleotide sequence tag (e.g. 3′ of the restriction endonuclease cleavage site).

The pool of defined tags ligated to linkers having the same sequence, or the two pools of defined nucleotide sequence tags ligated to linkers having different nucleotide sequences, were randomly ligated to each other “tail to tail”. The portion of the cDNA tag furthest from the linker is referred to as the “tail.” This created the ditag (ligated tag pair) having a first restriction endonuclease site upstream (5′) and a first restriction endonuclease site downstream (3′) of the ditag; a second restriction endonuclease cleavage site upstream and downstream of the ditag, and a linker oligonucleotide containing both a second restriction enzyme recognition site and an amplification primer hybridization site upstream and downstream of the ditag. In other words, the ditag is flanked by the first restriction endonuclease site, the second restriction endonuclease cleavage site and the linkers, respectively.

The ditag was amplified by utilizing primers for PCR which specifically hybridize to one strand of each linker. Cleavage of the amplified PCR product with the first restriction endonuclease allowed isolation of ditags which can then be concatenated by ligation. Analysis of the ditags or concatemers, whether or not amplification was performed, was performed by standard sequencing methods. After formation of concatemers, multiple tags can be cloned into a vector for sequence analysis, or alternatively, ditags or concatemers were directly sequenced without cloning by methods known to those of skill in the art.

The tags from a sequence were compared to a sequence database, for example using a computer method to match a sample sequence with known sequences.

### Computational Analysis

After the polynucleotide information is obtained, it is analyzed to identify polynucleotides that correspond to genes that are uniquely or differentially expressed between the two or more cell types. It is within the scope of this invention to perform the method described above using previously identified and stored sequence information that define and identify expressed genes. This information can be obtained from private, publically available and commercially available sequence databases.

For example, after a cell or tissue is selected for having a phenotype which is dependent on the presence of one gene product within a sample cell samples, e.g., cells that secrete a biological factor whose activity can be measured in an in vitro assay, cells that stain with an antibody that recognizes a specific antigen or cells that are lysed by cytotoxic T cells that recognize a specific antigen, the cells are further selected to identify sample cells that exhibit extremes of the chosen phenotype and ideally are matched in all other respects or phenotypic characteristics. For example, cells that are matched, e.g., from the same individual, would minimize having to deal with histocompatability differences

Ideally one selects two examples of sample cells (say “A” and “B”) that exhibit the chosen phenotype prominently and two examples of samples cells (say “C” and “D”) that do not have the phenotype at all. Using the method of this invention, polynucleotides present in a library form from each cell sample are isolated and their relative expression noted. The individual libraries are sequenced and the information regarding sequence and in some embodiments, relative expression, is stored in any functionally relevant program, e.g., in Compare Report using the SAGE software (available through Dr. Ken Kinzler at Johns Hopkins University). The Compare Report provides a tabulation of the polynucleotide sequences and their abundance for the samples (say A, B, C and D above) normalized to a defined number of polynucleotides per library (say 25,000). This is then imported into MS-ACCESS either directly or via copying the data into an Excel spreadsheet first and then into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of polynucleotides of specifically defined interest.

The researcher may create groups containing one or more project(s) by combining the counts of specific polynucleotides within a group (e.g., GroupNormal=Normal1+Normal2; GroupTumor=PrimaryTumor1+TumorCellLine). Additional characteristic values are also calculated for each tag in the group (e.g., average count, minimum count, maximum count). The researcher may calculate individual tag count ratios between groups, for example the ratio of the average GroupNormal count to the average GroupTumor count for each polynucleotide. The researcher may calculate a statistical measure of the significance of observed differences in tag counts between groups.

To identify the polynucleotides within MS-ACCESS, a query to sort polynucleotide tags based on their abundance in the sample cells is run. The output from the Query report lists specific polynucleotides (by sequence) that fit the sorting criteria and their abundance in the various sample cells.

The sorting is based on the principle that the gene product of interest (and hence the corresponding polynucleotide) is more abundant in the samples that prominently exhibit the chosen phenotype than in samples that do not exhibit the phenotype.

For example, one may query to identify polynucleotides that are present at a level of 10 or more in samples A and B and less than 1 in samples C and D, the results of the search might reveal that 5 different polynucleotides fit the sorting criteria hence there are 5 candidates genes to be tested to determine whether they confer the phenotype when transferred into samples like C and D that do not have the phenotype.

The more stringent the sorting criteria, the more efficient the sorting should be. Thus if one asked for polynucleotides that are at 5 copies or more in samples A and B and less than 5 copies in samples C and D, a large number of candidates would be generated. However, if one can increase the differential because the samples manifest extremes of the phenotype (say >10 in samples A and B and <1 in samples C and D) this restricts the number of candidates that will be identified.

Prior knowledge of what amount of gene product (hence abundance of polynucleotides) is required to confer the phenotype is not essential as one can arbitrarily select a set of sorting parameters, run the data analysis, and identify and test candidates. If the desired candidate is not found the stringency of the sorting criteria can be reduced (i.e. reduce the differential) and the new candidates that are found can be tested. Iterative cycles of sorting and testing candidates should eventually culminate in the successful recovery of the desired candidate.

TABLE 1

| | | | Number of |
|---|---|---|---|
| | Sorting | Number of | Candidates |
| Cycle | Criteria | Candidates | to Evaluate |
| 1 | ≧10 in | 10 | 10 |
| | samples A and B | | |
| | ≦1 in | | |
| | samples C and D | | |
| | (minimum | | |
| | differential = 10x) | | |
| 2 | ≧5 in | 30 | 20* |
| | samples A and B | | |
| | ≦2 in | | |
| | samples C and D | | |
| | (minimum | | |
| | differential = 2.5x) | | |
| 3 | ≧5 in | 80 | 50# |
| | samples A and B | | |
| | ≦5 in | | |
| | samples C and D | | |
| | (minimum | | |
| | differential = 1x) | | |

*Of the 30 candidates, 10 will have already been evaluated in cycle 1 so only 20 new candidates need to be evaluated
#Of the 80 candidates, 30 will have already been evaluated (10 in cycle 1, 20 in cycle 2) so only 50 need to be evaluated

Knowledge of what amount of gene product (hence abundance of polynucleotide) is required to confer the phenotype will permit the rationale use of stringent sorting criteria and greatly accelerate the search process as the desired gene may be captured within a handful of candidates

Establishing what amount of gene product is required to confer a specific phenotype will be dependent on the specific phenotype in question and the sensitivity of assays that measure that phenotype

For instance, the inventor has found that a frequency of 1/5000 (5 copies of a SAGE tag normalized to a library size of 25,000) correlates with sufficient expression of a tumor antigen within the sample cell to render it sensitive to lysis by an antigen specific T cell while a frequency of 1/25,000 correlates with the cell being weakly sensitive to lysis.

Thus, one could use a sorting criteria of ≧5 in samples cells that are susceptible to lysis and ≦1 in samples that are not susceptible to lysis to home in on a candidate tumor antigen.

Accordingly, one enters the individual polynucleotide sequences from the Query report into the program to determine if there is a match with any known genes or whether they are potentially novel (no match=NM).

One then retrieves cDNAs corresponding to specific sequences from the Query Report and test them individually in an appropriate biological assay to determine if they confer the phenotype. Of the candidates that correspond to known genes, it is a relatively easy task to obtain complementary DNAs for these candidates and test them individually to determine if they confer the specific phenotype in question when transferred into cells that do not exhibit the phenotype. If none of the known genes confer the phenotype, retrieve the cDNAs corresponding to the No Match sequences of the Query Report by PCR cloning and test the novel cDNAs individually for their ability to confer the phenotype. If the assumptions made up to this point are sound (i.e., a single gene product can confer the phenotype; the sorting criteria are not too stringent so as to exclude the desired candidate) then a cDNA corresponding to one of the candidates of the Query Report will be found to confer the phenotype and the search is over. If however none of the candidates are found to confer the phenotype then one may need to reduce the stringency of the sorting parameters to “cast a wider net” and capture more candidates to be tested as above.

In one embodiment, the polynucleotide or gene sequence can also be compared to a sequence database, for example, using a computer method to match a sample sequence with known sequences. Sequence identity can be determined by a sequence comparison using, i.e., sequence alignment programs that are known in the art, such as those described in CURRENT PROTOCOLS IN MOLECULAR BIOLOGY (F. M. Ausubel et al., eds., 1987) Supplement 30, section 7.7.18, Table 7.7.1. A preferred alignment program is ALIGN Plus (Scientific and Educational Software, Pennsylvania), preferably using default parameters, which are as follows: mismatch=2; open gap=0; and extend gap=2. Another preferred program is the BLAST program for alignment of two nucleotide sequences, using default parameters as follows: open gap=50; extension gap−2 penalties; gap×dropoff=0; expect=10; word size=11. The BLAST program is available at the following Internet address: http://www.ncbi.nlm.nih.gov.

Alternatively, the tag sequence can be translated into six reading frames; the predicted peptide sequences of all possible reading frames are then compared to individual sequences stored in a protein database. Parameters for determining the extent of homology set forth by one or more of the aforementioned alignment programs are well established in the art. They include but are not limited to p value and percent sequence identity. P value is the probability that the alignment is produced by chance. For a single alignment, the p value can be calculated according to Karlin et al. (1990) PNAS 87: 2246. For multiple alignments, the p value can be calculated using a heuristic approach such as the one programmed in Blast. Percent sequence identify is defined by the ratio of the number of nucleotide or amino acid matches between the query sequence and the known sequence when the two are optimally aligned. A tag sequence is considered to lack substantial homology with any known sequences when the regions of alignment of comparable length exhibit less than 30% of sequence identity, more preferably less than 20% identity, even more preferably less than 10% identity.

### Identification of Larger Fragments and Open Reading Frames

Uniquely expressed or overexpressed tags in the target cells as compared to the control cells are putative antigens for use in the methods of this invention. Five methods are disclosed herein which allow one of skill in the art to isolate a larger polynucleotide, gene or cDNA containing or corresponding to the tags of interest.

### RACE-PCR Technique

One method to isolate the gene or cDNA which code for a polypeptide or protein and which corresponds to a transcript of this invention, involves the 5′-RACE-PCR technique. In this technique, the poly-A mRNA that contains the coding sequence of particular interest is first identified by hybridization to a sequence disclosed herein and then reverse transcribed with a 3′-primer comprising the sequence disclosed herein. The newly synthesized cDNA strand is then tagged with an anchor primer of a known sequence, which preferably contains a convenient cloning restriction site attached at the 5′end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc, Clotech) according to the manufacturer's instructions.

### Identification of Known Genes or ESTs

In addition, databases exist that reduce the complexity of ESTs by assembling contiguous EST sequences into tentative genes. For example, TIGR has assembled human ESTs into a datable called THC for tentative human consensus sequences. The THC database allows for a more definitive assignment compared to ESTs alone. Software programs exist (TIGR assembler and TIGEM EST assembly machine and contig assembly program (see Huang, X. (1996) Genomics 33:21-23)) that allow for assembling ESTs into contiguous sequences from any organism.

### Isolation of cDNAs from a Library by Probing with the SAGE Transcript or Tag

Alternatively, mRNA from a sample preparation is used to construct cDNA library in the ZAP Express vector following the procedure described in Velculescu, et al. (1997) Science 270:484. The ZAP Express cDNA synthesis kit (Stratagene) is used accordingly to the manufacturer's protocol. Plates containing 250 to 2000 plaques are hybridized as described in Rupert, et al. (1988) Mol. Cell. Bio. 8:3104 to oligonucleotide probes with the same conditions previously described for standard probes except that the hybridization temperature is reduced to room temperature. Washes are performed in 6× standard-saline-citrate 0.1% SDS for 30 minutes at room temperature. The probes are labeled with ³²P-ATP through use of T4 polynucleotide kinase.

### Isolation of Partial cDNA (3′ fragment) by 3′ Directed PCR Reaction

This procedure is a modification of the protocol described in Polyak, et al. (1997) Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source believed to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 5′-B-TCC GGC GCG CCG TTT T CC CAG TCA CGA(30)-3′ (SEQ ID NO: 1), contains a poly-T stretch at the 3′ end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the streptavidin-coated magnetic beads. Theoretically, any sufficiently-sized DNA region capable of hybridizing to a PCR primer can be used as well as any other 8 base pair recognizing endonuclease. cDNA constructed utilizing this or similar modified oligo-dT primer is then processed exactly as described in U.S. Pat. No. 5,695,937 up until adapter ligation where only one adapter is ligated to the cDNA pool. After adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

### Isolation of cDNA Using GeneTrapper or Modified GeneTrapper Technology

The reagents and manufacturer's instructions for this technology are commercially available from Life Technologies, Inc., Gaithersburg, Md. Briefly, a complex population of single-stranded phagemid DNA containing directional cDNA inserts is enriched for the target sequence by hybridization in solution to a biotinylated oligonucleotide probe complementary to the target sequence. The hybrids are captured on streptavidin-coated paramagnetic beads. A magnet retrieves the paramagnetic beads from the solution, leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded target to double-stranded DNA. Following transformation and plating, typically 20% to 100% of the colonies represent the cDNA clone of interest. To identify the desired cDNA clone, the colonies may be screened by colony hybridization using the ³²P-labeled oligonucleotide as described above for solution hybridization, or alternatively by DNA sequencing and alignment of all sequences obtained from numerous clones to determine a consensus sequence.

### Confirmation of Immunogenicity

The genes or gene fragments identified as putative antigens are isolated and expressed in appropriate host vector systems for recombinant production of antigen and use in methods to confirm immunogenicity. These methods are described below.

### Delivery Vehicles Comprising a Polynucleotides

Polynucleotides encoding the antigens can be delivered to cells in a variety of gene delivery vehicles. A polynucleotide of the invention can be contained within a cloning or expression vector. These vectors (especially expression vectors) can in turn be manipulated to assume any of a number of forms which may, for example, facilitate delivery to and/or entry into a cell.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a procaryotic or a eucaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook, et al. (1989) Supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook et al. (1989) Supra for this methodology. Thus, this invention also provides a host cell, e.g. a mammalian cell, an animal cell (rat or mouse), a human cell, or a procaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6. Miller et al. (1989) BioTechniques 7:980-990. The methodology of using replication-incompetent retroviruses for retroviral-mediated gene transfer of gene markers is well established. Correll et al. (1989) PNAS 86:8912; Bordignon (1989) PNAS 86:8912-52; Culver (1991) PNAS 88:3155; and Rill (1991) Blood 79(10):2694-700.

In general, genetic modifications of cells employed in the present invention are accomplished by introducing a vector containing a polynucleotide comprising sequences encoding an peptide of the invention. A variety of different gene transfer vectors, including viral as well as non-viral systems can be used.

A wide variety of non-viral vehicles for delivery of a polynucleotide of the invention are known in the art and are encompassed in the present invention. A polynucleotide of the invention can be delivered to a cell as naked DNA. WO 97/40163. Alternatively, a polynucleotide of the invention can be delivered to a cell associated in a variety of ways with a variety of substances (forms of delivery) including, but not limited to cationic lipids; biocompatible polymers, including natural polymers and polymers; lipoproteins; polypeptides; polysaccharides; lipopolysaccharides; artificial viral envelopes; metal particles; and bacteria. A delivery vehicle may take the form of a microparticle. Mixtures or conjugates of these various substances can also be used as delivery vehicles. A polynucleotide of the invention can be associated with these various forms of delivery non-covalently or covalently.

Included in the non-viral vector category are prokaryotic plasmids and eukaryotic plasmids. Non-viral vectors (i.e., cloning and expression vectors) having cloned therein a polynucleotide(s) of the invention can be used for expression of recombinant polypeptides as well as a source of polynucleotide of the invention. Cloning vectors can be used to obtain replicate copies of the polynucleotides they contain, or as a means of storing the polynucleotides in a depository for future recovery. Expression vectors (and host cells containing these expression vectors) can be used to obtain polypeptides produced from the polynucleotides they contain. They may also be used where it is desirable to express polypeptides, encoded by an operably linked polynucleotide, in an individual, such as for eliciting an immune response via the polypeptide(s) encoded in the expression vector(s). Suitable cloning and expression vectors include any known in the art, e.g., those for use in bacterial, mammalian, yeast and insect expression systems. Specific vectors and suitable host cells are known in the art and need not be described in detail herein. For example, see Gacesa and Ramji, Vectors, John Wiley & Sons (1994).

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins substances, e.g., ampicillin, neomycyin, methotrexate, etc.; (b) complement auxotrophic deficiencies; or (c) supply critical nutrients not available from complex media. The choice of the proper marker gene will depend on the host cell, and appropriate genes for different hosts are known in the art. Cloning and expression vectors also typically contain a replication system recognized by the host.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen. The Examples provided herein also provide examples of cloning vectors.

Expression vectors generally are replicable polynucleotide constructs that contain a polynucleotide encoding a polypeptide of interest. The polynucleotide encoding the polypeptide of interest is operably linked to suitable transcriptional controlling elements, such as promoters, enhancers and terminators. For expression (i.e., translation), one or more translational controlling elements are also usually required, such as ribosome binding sites, translation initiation sites, and stop codons. A polynucleotide sequence encoding a signal peptide can also be included to allow a polypeptide, encoded by an operably linked polynucleotide, to cross and/or lodge in cell membranes or be secreted from the cell. A number of expression vectors suitable for expression in eukaryotic cells including yeast, avian, and mammalian cells are known in the art. Examples of mammalian expression vectors contain both prokaryotic sequence to facilitate the propagation of the vector in bacteria, and one or more eukaryotic transcription units that are expressed in eukaryotic cells. Examples of mammalian expression vectors suitable for transfection of eukaryotic cells include the pcDNAI/amp, pcDNAI/neo, pRc/CMV, pSV2gpt, pSV2neo, pRSVneo, and pHyg derived vectors. Alternatively, derivatives of viruses such as the bovine papilloma virus (BPV-1), or Epstein-Barr virus (pHEB, pREP derived vectors) can be used for expression in mammalian cells. Examples of expression vectors for yeast systems, include YEP24, YIP5, YEP51, YEP52, YES2 and YRP17, which are cloning and expression vehicles useful for introduction of constructs into S. cerevisiae. Broach et al. (1983) EXPERIMENTAL MANIPULATION OF GENE EXPRESSION, ed. M. Inouye, Academic Press. p. 83. Baculovirus expression vectors for expression in insect cells include pVL-derived vectors (such as pVL1392, pVL1393 and pVL941), pAcUW-derived vectors and pBlueBac-derived vectors.

Viral vectors include, but are not limited to, DNA viral vectors such as those based on adenoviruses, herpes simplex virus, poxviruses such as vaccinia virus, and parvoviruses, including adeno-associated virus; and RNA viral vectors, including, but not limited to, the retroviral vectors. Retroviral vectors include murine leukemia virus, and lentiviruses such as human immunodeficiency virus. Naldini et al. (1996) Science 272:263-267.

Replication-defective retroviral vectors harboring a polynucleotide of the invention as part of the retroviral genome can be used. Such vectors have been described in detail. (Miller, et al. (1990) Mol. Cell Biol. 10:4239; Kolberg, R. (1992) J. NIH Res. 4:43; Cornetta, et al. (1991) Hum. Gene Therapy 2:215).

Adenovirus and adeno-associated virus vectors useful in the genetic modifications of this invention may be produced according to methods already taught in the art. (See, e.g., Karlsson, et al. (1986) EMBO 5:2377; Carter (1992) Current Opinion in Biotechnology 3:533-539; Muzcyzka (1992) Current Top. Microbiol. Immunol. 158:97-129; GENE TARGETING: A PRACTICAL APPROACH (1992) ed. A. L. Joyner, Oxford University Press, NY). Several different approaches are feasible.

Additional references describing viral vectors which could be used in the methods of the present invention include the following: Horwitz, M. S., Adenoviridae and Their Replication, in Fields, B., et al. (eds.) VIROLOGY, Vol. 2, Raven Press New York, pp. 1679-1721, 1990); Graham, F. et al., pp. 109-128 in METHODS IN MOLECULAR BIOLOGY, VOL. 7: GENE TRANSFER AND EXPRESSION PROTOCOLS, Murray, E. (ed.), Humana Press, Clifton, N.J. (1991); Miller, et al. (1995) FASEB Journal 9:190-199, Schreier (1994) Pharmaceutica Acta Helvetiae 68:145-159; Schneider and French (1993) Circulation 88:1937-1942; Curiel, et al. (1992) Human Gene Therapy 3:147-154; Graham, et al., WO 95/00655 (Jan. 5, 1995); Falck-Pedersen WO 95/16772 (Jun. 22, 1995); Denefle, et al. WO 95/23867 (Sep. 8, 1995); Haddada, et al. WO 94/26914 (Nov. 24, 1994); Perricaudet, et al. WO 95/02697 (Jan. 26, 1995); and Zhang, et al. WO 95/25071 (Oct. 12, 1995).

### Databases and High Through-put Screens

The sequences of polynucleotides of this invention also can be used for comparison to known and unknown sequences using a computer-based method to match a sample sequence with known sequences. Thus, this invention also provides the sequences of the polynucleotides of this invention in a computer database or in computer readable form, including applications utilizing the internet.

A linear search through such a database may be used. Alternatively, the polynucleotide sequence can be converted into a unique numeric representation. The comparison aspects may be implemented in hardware or software, or a combination of both. Preferably, these aspects of the invention are implemented in computer programs executing on a programable computer comprising a processor, a data storage system (including volatile and non-volatile memory and/or storage elements), at least one input device, and at least one output device. Data input through one or more input devices for temporary or permanent storage in the data storage system includes sequences, and may include previously generated polynucleotides and codes for known and/or unknown sequences. Program code is applied to the input data to perform the functions described above and generate output information. The output information is applied to one or more output devices, in known fashion.

Each such computer program is preferably stored on a storage media or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

A polynucleotide of the invention also can be attached to a solid support for use in high throughput screening assays. PCT WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat. Nos. 5,578,832; and 5,631,734. The obtained measurement is directly correlated with gene expression level.

Results from the chip assay are typically analyzed using a computer software program. See, for example, EP 717,113 A2 and WO 95/20681. The hybridization data is read into the program, which calculates the expression level of the targeted gene(s). This figure is compared against existing data sets of gene expression levels for that cell type.

For example, the database and methods of using the database provides a means to differentiate expression levels and identify novel peptides. Alternatively, the database and methods can be used to distinguish a normal cell (in this case, the reference cell) from a neoplastic cell (i.e., the test cell). It also allows one to differentiate between neoplastic cells biopsied from different regions from a patient or different subjects or gene expression before or after treatment with a potential therapeutic agent. It can be used to analyze drug toxicity and efficacy, as well as to selectively look at protein categories which are expected to be affected by a drug or which may be overexpressed as a result of treatment with a drug, such as the various multi-drug resistant genes. Additional utilities of the database include, but are not limited to analysis of the developmental state of a test cell, the influence of viral or bacterial infection, control of cell cycle, effect of a tumor suppressor gene or lack thereof, polymorphism within the cell type, apoptosis, and the effect of regulatory genes.

### Host Cells Comprising Polynucleotides of the Invention

The present invention further provides host cells comprising polynucleotides of the invention. Host cells containing the polynucleotides of this invention are useful for the recombinant replication of the polynucleotides and for the recombinant production of peptides of the invention. Alternatively, host cells comprising a polynucleotide of the invention may be used to induce an immune response in a subject in the methods described herein.

Host cells which are suitable for recombinant replication of the polynucleotides of the invention, and for the recombinant production of peptides of the invention can be prokaryotic or eukaryotic. Host systems are known in the art and need not be described in detail herein. Prokaryotic hosts include bacterial cells, for example E. coli, B. subtilis, and mycobacteria. Among eukaryotic hosts are yeast, insect, avian, plant, C. elegans (or nematode) and mammalian cells. These cells are cultured in conventional nutrient media modified as appropriate for inducing promoters, selecting transformants, or amplifying the genes encoding the desired sequences.

When the host cells are antigen presenting cells, they can be used as cancer vaccine or to expand a population of immune effector cells such as tumor infiltrating lymphocytes which in turn are useful in adoptive immunotherapies.

In some of these embodiments, isolated host cells are APCs. APCs include, but are not limited to, dendritic cells (DCs), monocytes/macrophages, B lymphocytes or other cell type(s) expressing the necessary MHC/co-stimulatory molecules.

The efficiency of transduction of DCs or other APCs can be assessed by immunofluorescence using fluorescent antibodies specific for the tumor antigen being expressed (Kim, et al. (1997) J. Immunother. 20:276-286). Alternatively, the antibodies can be conjugated to an enzyme (e.g. HRP) giving rise to a colored product upon reaction with the substrate. The actual amount of antigenic polypeptides being expressed by the APCs can be evaluated by ELISA.

In vivo transduction of DCs, or other APCs, can be accomplished by administration of a viral vectors comprising a polynucleotide of the invention via different routes including intravenous, intramuscular, intranasal, intraperitoneal or cutaneous delivery. One method which can be used is cutaneous delivery of Ad vector at multiple sites using a total dose of approximately 1×10¹⁰−1×10¹² i.u. Levels of in vivo transduction can be roughly assessed by co-staining with antibodies directed against APC marker(s) and the peptide epitope being expressed. The staining procedure can be carried out on biopsy samples from the site of administration or on cells from draining lymph nodes or other organs where APCs (in particular DCs) may have migrated. Condon et al. (1996) Nature Med. 2:1122-1128; Wan et al. (1997) Human Gene Therapy 8:1355-1363. The amount of antigen being expressed at the site of injection or in other organs where transduced APCs may have migrated can be evaluated by ELISA on tissue homogenates.

APCs can also be transduced in vitro/ex vivo by non-viral gene delivery methods such as electroporation, calcium phosphate precipitation or cationic lipid/plasmid DNA complexes. Arthur et al. (1997) Cancer Gene Therapy 4:17-25. Transduced APCs can subsequently be administered to the host via an intravenous, subcutaneous, intranasal, intramuscular or intraperitoneal route of delivery.

In vivo transduction of DCs, or other APCs, can potentially be accomplished by administration of cationic lipid/plasmid DNA complexes delivered via the intravenous, intramuscular, intranasal, intraperitoneal or cutaneous route of administration. Gene gun delivery or injection of naked plasmid DNA into the skin also leads to transduction of DCs. Condon et al. (1996) Nature Med. 2:1122-1128; Raz et al. (1994) PNAS 91:9519-9523. Intramuscular delivery of plasmid DNA may also be used for immunization. Rosato et al. (1997) Human Gene Therapy 8:1451-1458.

The transduction efficiency and levels of transgene expression can be assessed as described above for viral vectors.

In some embodiments, the immune effector cells and/or the APCs are genetically modified. Using standard gene transfer, genes coding for co-stimulatory molecules and/or stimulatory cytokines can be inserted prior to, concurrent to or subsequent to expansion of the immune effector cells.

### Antibodies

Also provided by this invention is an antibody capable of specifically forming a complex with a peptide(s) and/or polypeptide(s) of this invention. The term “antibody” includes polyclonal antibodies and monoclonal antibodies. The antibodies include, but are not limited to mouse, rat, and rabbit or human antibodies. The antibodies are useful to identify and purify peptides/polypeptides of the invention and APCs expressing the peptides/polypeptides. They also are useful to inhibit the activation of T cell by the antigens of the invention. Accordingly, methods of inhibiting T cell activation by contacting a T cell with an effective amount of antibody raised against the antigen is further provided by this invention. These methods can be conducted in vitro, in vivo and ex vivo.

Laboratory methods for producing polyclonal antibodies and monoclonal antibodies, as well as deducing their corresponding nucleic acid sequences, are known in the art, see Harlow and Lane (1988) Supra and Sambrook, et al. (1989) Supra. The monoclonal antibodies of this invention can be biologically produced by introducing protein or a fragment thereof into an animal, e.g., a mouse or a rabbit. The antibody producing cells in the animal are isolated and fused with myeloma cells or heteromyeloma cells to produce hybrid cells or hybridomas. Accordingly, the hybridoma cells producing the monoclonal antibodies of this invention also are provided.

Thus, using the protein or fragment thereof, and well known methods, one of skill in the art can produce and screen the hybridoma cells and antibodies of this invention for antibodies having the ability to bind the proteins or polypeptides.

If a monoclonal antibody being tested binds with the peptide or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the peptide or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al. (1985) PNAS 82:8653 or Spira, et al. (1984) J. Immunol. Methods 74:307.

This invention also provides biological active fragments of the polyclonal and monoclonal antibodies described above. These “antibody fragments” retain some ability to selectively bind with its antigen or immunogen. Such antibody fragments can include, but are not limited to: (1) Fab, (2) Fab′ (3) F(ab′)₂, (4)Fv, and (5) SCA (single chain antibody).

A specific example of “a biologically active antibody fragment” is a CDR region of the antibody. Methods of making these fragments are known in the art, see for example, Harlow and Lane (1988) Supra.

The antibodies of this invention also can be modified to create chimeric antibodies and humanized antibodies (Oi et al. (1986) BioTechniques 4(3):214). Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies. Herlyn, et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas suggests that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody can have a binding domain in the hypervariable region which is the mirror image of the epitope bound by the first monoclonal antibody. Thus, in this instance, the anti-idiotypic monoclonal antibody could be used for immunization for production of these antibodies. “Epitope” refers to that portion of a molecule which is specifically recognized by an antibody or a T cell antigen receptor. It is also referred to as an “antigenic determinant” or an “antigenic region”. Epitopic determinants usually consist of chemically active surface groupings of molecules such as amino acids or sugar side chains and usually have specific three dimensional structural characteristics, as well as specific charge characteristics.

The antibodies of this invention can be linked to a detectable agent or label. There are many different labels and methods of labeling known to those of ordinary skill in the art.

The coupling of antibodies to low molecular weight haptens can increase the sensitivity of the assay. The haptens can then be specifically detected by means of a second reaction. For example, it is common to use haptens such as biotin (which reacts with avidin), or dinitrophenyl, pyridoxal, and fluorescein, which can react with specific anti-hapten antibodies. See Harlow and Lane (1988) Supra.

The monoclonal antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

### Host Cells Comprising Antigenic Peptides

The invention further provides isolated host cells comprising antigenic peptides of the invention. In some embodiments, these host cells present one or more peptides of the invention on the surface of the cell in the context of an MHC molecule, i.e., an antigenic peptide of the invention is bound to a cell surface MHC molecule such that the peptide can be recognized by an immune effector cell. Isolated host cells which present the polypeptides of this invention in the context of MHC molecules are useful as cancer vaccines or are further useful to expand and isolate a population of educated, antigen-specific immune effector cells. The immune effector cells, e.g., cytotoxic T lymphocytes, are produced by culturing naïve immune effector cells with antigen-presenting cells cells which present the polypeptides in the context of MHC molecules on the surface of the APCs. The population can be purified using methods known in the art, e.g., FACS analysis or FICOLL™ gradient. The methods to generate and culture the immune effector cells as well as the populations produced thereby also are the inventor's contribution and invention. Pharmaceutical compositions comprising the cells and pharmaceutically acceptable carriers are useful in adoptive immunotherapy. Prior to administration in vivo, the immune effector cells are screened in vitro for their ability to lyse the target cells.

### Antigen-presenting Matrices Comprising Peptides

An antigenic epitope of the invention can be presented (bound by) an MUC Class I or Class II molecule in an antigen-presenting matrix, with or without co-stimulatory molecules necessary for CD4+ or CD8+ T cell activation. Whether co-stimulatory molecules are present may depend on the intended use of the antigen-presenting matrix.

Antigen-presenting matrices include those on the surface of an APC as well as synthetic antigen-presenting matrices. Antigen-presenting matrices are a form of solid support. APCs suitable for use in the present invention are capable of presenting exogenous peptide or protein or endogenous antigen to T cells in association with an antigen-presenting molecule, such as an MHC molecule. APCs include, but are not limited to, macrophages, dendritic cells, CD40-activated B cells, antigen-specific B cells, tumor cells, virus-infected cells, and genetically modified cells.

APCs can obtained from a variety of sources, including but not limited to, peripheral blood mononuclear cells (PBMC), whole blood or fractions thereof containing mixed populations, spleen cells, bone marrow cells, tumor infiltrating lymphocytes, cells obtained by leukapheresis, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers. APC's can also be treated in vitro with one or more biological modifiers.

The APCs are generally alive but can also be irradiated, mitomycin C treated, attenuated, or chemically fixed. Further, the APCs need not be whole cells. Instead, vesicle preparations of APCs can be used.

APCs can be genetically modified, i.e., transfected with a recombinant polynucleotide construct such that they express a polypeptide or an RNA molecule which they would not normally express or would normally express at lower levels. Examples of polynucleotides include, but are not limited to, those which encode an MHC molecule; a co-stimulatory molecule such as B7; and a peptide or polypeptide of the invention.

Cells which do not normally function in vivo in mammals as APCs can be modified in such a way that they function as APCs. A wide variety of cells can function as APCs when appropriately modified. Examples of such cells are insect cells, for example Drosophila or Spodoptera; and foster cells, such as the human cell line T2. For example, expression vectors which direct the synthesis of one or more antigen-presenting polypeptides, such as MHC molecules, optionally also accessory molecules such as B7, can be introduced into these cells to effect the expression on the surface of these cells antigen presentation molecules and, optionally, accessory molecules or functional portions thereof. Alternatively, antigen-presenting polypeptides and accessory molecules which can insert themselves into the cell membrane can be used. For example, glycosyl-phosphotidylinositol (GPI)-modified polypeptides can insert themselves into the membranes of cells. Hirose et al. (1995) Methods Enzymol. 250:582-614; and Huang et al. (1994) Immunity 1:607-613. Accessory molecules include, but are not limited to, co-stimulatory antibodies such as antibodies specific for CD28, CD80, or CD86; costimulatory molecules, including, but not limited to, B7.1 and B7.2; adhesion molecules such as ICAM-1 and LFA-3; and survival molecules such as Fas ligand and CD70. See, for example, PCT Publication No. WO 97/46256.

Foster antigen presenting cells are particularly useful as APCs. Foster APCs are derived from the human cell line 174xCEM.T2, referred to as T2, which contains a mutation in its antigen processing pathway that restricts the association of endogenous peptides with cell surface MHC class I molecules. Zweerink et al. (1993) J. Immunol. 150:1763-1771. This is due to a large homozygous deletion in the MHC class II region encompassing the genes TAP1, TAP2, LMP1, and LMP2, which are required for antigen presentation to MHC class 1-restricted CD8⁺ CTLs. In effect, only “empty” MHC class I molecules are presented on the surface of these cells. Exogenous peptide added to the culture medium binds to these MHC molecules provided that the peptide contains the allele-specific binding motif. These T2 cells are referred to herein as “foster” APCs. They can be used in conjunction with this invention to present antigen(s).

Transduction of T2 cells with specific recombinant MHC alleles allows for redirection of the MHC restriction profile. Libraries tailored to the recombinant allele will be preferentially presented by them because the anchor residues will prevent efficient binding to the endogenous allele.

High level expression of MHC molecules makes the APC more visible to the CTLs. Expressing the MHC allele of interest in T2 cells using a powerful transcriptional promoter (e.g., the CMV promoter) results in a more reactive APC (most likely due to a higher concentration of reactive MHC-peptide complexes on the cell surface).

Alternatively, a synthetic antigen-presenting matrix can be used to present antigen to an effector cell(s). A synthetic matrix can include an antigen presenting molecule, preferably an MHC Class I or MHC Class II molecule, immobilized on a solid support, for example, beads or plates. Accessory molecules can be present, which can be co-immobilized or soluble, the molecules including, but not limited to, co-stimulatory antibodies such as antibodies specific for CD28, CD80, or CD86; costimulatory molecules, including, but not limited to, B7.1 and B7.2; adhesion molecules such as ICAM-1 and LFA-3; and survival molecules such as Fas ligand and CD70. Portions of accessory molecules can also be used, as long as their function is maintained. Solid supports include metals or plastics, porous materials, microbeads, microtiter plates, red blood cells, and liposomes. See, for example, International Patent Publication Nos. WO 97/46256; and WO 97/35035.

Methods for determining whether an antigen-presenting matrix, whether it is on a cell surface or on a synthetic support, is capable of presenting antigen to an immune effector cell in such a manner as to effect activation of the immune effector cell, are known in the art and include, for example,³H-thymidine uptake by effector cells, cytokine production by effector cells, and cytolytic ⁵¹Cr-release assays.

In some embodiments, an antigenic peptide of the invention is presented on an antigen-presenting matrix in a Class I or Class II MHC molecule such that the peptide is bound by a TCR on a CD4+ or CD8+ T cell, but the antigen-presenting matrix lacks one or more co-stimulatory molecules required for activation of the T cell. These antigen-presenting matrices induce T cell anergy (unresponsiveness), and are useful in methods described herein for reducing or suppressing an immune response. Methods for determining whether an antigen-presenting matrix is capable of presenting antigen to an immune effector cell, in such a manner as to effect T cell anergy, are known in the art.

The following is a brief description of two fundamental approaches for the isolation of APC. These approaches involve (1) isolating bone marrow precursor cells (CD34⁺) from blood and stimulating them to differentiate into APC; or (2) collecting the precommitted APCs from peripheral blood. In the first approach, the patient must be treated with cytokines such as GM-CSF to boost the number of circulating CD34⁺ stem cells in the peripheral blood.

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/nonadherence steps (Freudenthal et al. (1990) PNAS 87:7698-7702); Percoll gradient separations (Mehta-Damani et al. (1994) J. Immunol. 153:996-1003); and fluorescence activated cell sorting techniques (Thomas et al. (1993) J. Immunol. 151:6840-52).

One technique for separating large numbers of cells from one another is known as countercurrent centrifugal elutriation (CCE). In this technique, cells are subject to simultaneous centrifugation and a washout stream of buffer which is constantly increasing in flow rate. The constantly increasing countercurrent flow of buffer leads to fractional cell separations that are largely based on cell size.

In one aspect of the invention, the APC are precomnmitted or mature dendritic cells which can be isolated from the white blood cell fraction of a mammal, such as a murine, simian or a human (See, e.g., WO 96/23060). The white blood cell fraction can be from the peripheral blood of the mammal. This method includes the following steps: (a) providing a white blood cell fraction obtained from a mammalian source by methods known in the art such as leukopheresis; (b) separating the white blood cell fraction of step (a) into four or more subfractions by countercurrent centrifugal elutriation, (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c), and (e) collecting the enriched fraction of step (d), preferably at about 4° C. One way to identify the dendritic cell-enriched fraction is by fluorescence-activated cell sorting. The white blood cell fraction can be treated with calcium ionophore in the presence of other cytokines, such as recombinant (rh) rhIL-12, rhGM-CSF, or rhIL-4. The cells of the white blood cell fraction can be washed in buffer and suspended in Ca⁺⁺/Mg⁺⁺ free media prior to the separating step. The white blood cell fraction can be obtained by leukopheresis. The dendritic cells can be identified by the presence of at least one of the following markers: HLA-DR, HLA-DQ, or B7. 2, and the simultaneous absence of the following markers: CD3, CD14, CD16, 56, 57, and CD 19, 20. Monoclonal antibodies specific to these cell surface markers are commercially available.

More specifically, the method requires collecting an enriched collection of white cells and platelets from leukopheresis that is then further fractionated by countercurrent centrifugal elutriation (CCE). Abrahamsen et al. (1991) J. Clin. Apheresis. 6:48-53. Cell samples are placed in a special elutriation rotor. The rotor is then spun at a constant speed of, for example, 3000 rpm. Once the rotor has reached the desired speed, pressurized air is used to control the flow rate of cells. Cells in the elutriator are subjected to simultaneous centrifugation and a washout stream of buffer which is constantly increasing in flow rate. This results in fractional cell separations based largely but not exclusively on differences in cell size.

Quality control of APC and more specifically DC collection and confirmation of their successful activation in culture is dependent upon a simultaneous multi-color FACS analysis technique which monitors both monocytes and the dendritic cell subpopulation as well as possible contaminant T lymphocytes. It is based upon the fact that DCs do not express the following markers: CD3 (T cell); CD14 (monocyte); CD16, 56, 57 (NK/LAK cells); CD19, 20 (B cells). At the same time, DCs do express large quantities of HLA-DR, significant HLA-DQ and B7.2 (but little or no B7.1) at the time they are circulating in the blood (in addition they express Leu M7 and M9, myeloid markers which are also expressed by monocytes and neutrophils).

Once collected, the DC rich/monocyte APC fractions (usually 150 through 190) can be pooled and cryopreserved for future use, or immediately placed in short term culture.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 24-48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2.

Specific combination(s) of cytokines have been used successfully to amplify (or partially substitute) for the activation/conversion achieved with calcium ionophore: these cytokines include but are not limited to purified or recombinant human (“rh”) rhGM-CSF, rhWL-2, and rhWL-4. Each cytokine when given alone is inadequate for optimal upregulation.

### Immune Effector Cells

The present invention makes use of the above-described antigen-presenting matrices, including APCs, to stimulate production of an enriched population of antigen-specific immune effector cells. Accordingly, the present invention provides a population of cells enriched in educated, antigen-specific immune effector cells, specific for an antigenic peptide of the invention. These cells can cross-react with (bind specifically to) antigenic determinants (epitopes) on natural (endogenous) antigens. In some embodiments, the natural antigen is on the surface of tumor cells and the educated, antigen-specific immune effector cells of the invention suppress growth of the tumor cells. When APCs are used, the antigen-specific immune effector cells are expanded at the expense of the APCs, which die in the culture. The process by which naïve immune effector cells become educated by other cells is described essentially in Coulie (1997) Molec. Med. Today 3:261-268.

The APCs prepared as described above are mixed with naïve immune effector cells. Preferably, the cells may be cultured in the presence of a cytokine, for example IL2. Because dendritic cells secrete potent immunostimulatory cytokines, such as IL-12, it may not be necessary to add supplemental cytokines during the first and successive rounds of expansion. In any event, the culture conditions are such that the antigen-specific immune effector cells expand (i.e. proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding for example, IL-2, IL-11 or IL-13. Methods for introducing transgenes in vitro, ex vivo and in vivo are well known in the art. See Sambrook, et al. (1989) Supra.

An effector cell population suitable for use in the methods of the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69⁺ cells, or inactivating alloreactive cells, or inducing anergy in the alloreactive cell population.

Hybrid immune effector cells can also be used. Immune effector cell hybrids are known in the art and have been described in various publications. See, for example, International Patent Application Nos. WO 98/46785; and WO 95/16775.

The effector cell population can comprise unseparated cells, i.e., a mixed population, for example, a PBMC population, whole blood, and the like. The effector cell population can be manipulated by positive selection based on expression of cell surface markers, negative selection based on expression of cell surface markers, stimulation with one or more antigens in vitro or in vivo, treatment with one or more biological modifiers in vitro or in vivo, subtractive stimulation with one or more antigens or biological modifiers, or a combination of any or all of these.

Effector cells can obtained from a variety of sources, including but not limited to, PBMC, whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers.

Methods of extracting and culturing effector cells are well known. For example, effector cells can be obtained by leukapheresis, mechanical apheresis using a continuous flow cell separator. For example, lymphocytes and monocytes can be isolated from the buffy coat by any known method, including, but not limited to, separation over Ficoll-Hypaque™ gradient, separation over a Percoll gradient, or elutriation. The concentration of Ficoll-Hypaque™ can be adjusted to obtain the desired population, for example, a population enriched in T cells. Other methods based on affinity are known and can be used. These include, for example, fluorescence-activated cell sorting (FACS), cell adhesion, magnetic bead separation, and the like. Affinity-based methods may utilize antibodies, or portions thereof, which are specific for cell-surface markers and which are available from a variety of commercial sources, including, the American Type Culture Collection (Manassas, Md.). Affinity-based methods can alternatively utilize ligands or ligand analogs, of cell surface receptors.

The effector cell population can be subjected to one or more separation protocols based on the expression of cell surface markers. For example, the cells can be subjected to positive selection on the basis of expression of one or more cell surface polypeptides, including, but not limited to, “cluster of differentiation” cell surface markers such as CD2, CD3, CD4, CD8, TCR, CD45, CD45RO, CD45RA, CD11b, CD26, CD27, CD28, CD29, CD30, CD31, CD40L; other markers associated with lymphocyte activation, such as the lymphocyte activation gene 3 product (LAG3), signaling lymphocyte activation molecule (SLAM), T1/ST2; chemokine receptors such as CCR3, CCR4, CXCR3, CCR5; homing receptors such as CD62L, CD44, CLA, CD146, a4b7, aEb7; activation markers such as CD25, CD69 and OX40; and lipoglycans presented by CD1. The effector cell population can be subjected to negative selection for depletion of non-T cells and/or particular T cell subsets. Negative selection can be performed on the basis of cell surface expression of a variety of molecules, including, but not limited to, B cell markers such as CD19, and CD20; monocyte marker CD 14; the NK cell marker CD56.

An effector cell population can be manipulated by exposure, in vivo or in vitro, to one or more biological modifiers. Suitable biological modifiers include, but are not limited to, cytokines such as IL-2, IL-4, IL-10, TNF-α, IL-12, IFN-γ; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL2 receptor, anti-CD28; chemokines, including, for example, lymphotactin. The biological modifiers can be native factors obtained from natural sources, factors produced by recombinant DNA technology, chemically synthesized polypeptides or other molecules, or any derivative having the functional activity of the native factor. If more than one biological modifier is used, the exposure can be simultaneous or sequential.

The present invention provides compositions comprising immune effector cells, which may be T cells, enriched in antigen-specific cells, specific for a peptide of the invention. By “enriched” is meant that a cell population is at least about 50-fold, more preferably at least about 500-fold, and even more preferably at least about 5000-fold or more enriched from an original naïve cell population. The proportion of the enriched cell population which comprises antigen-specific cells can vary substantially, from less than 10% up to 100% antigen-specific cells. If the cell population comprises at least 50%, preferably at least 70%, more preferably at least 80%, and even more preferably at least 90%, antigen-specific immune effector cells, specific for a peptide of the invention, then the population is said to be “substantially pure”. The percentage which are antigen-specific can readily be determined, for example, by a ³H-thymidine uptake assay in which the effector cell population (for example, a T-cell population) is challenged by an antigen-presenting matrix presenting an antigenic peptide of the invention.

### Compositions of the Invention

This invention also provides compositions containing any of the above-mentioned peptides, polypeptides, polynucleotides, antigen-presenting matrices, vectors, cells, antibodies and fragments thereof, and an acceptable solid or liquid carrier. When the compositions are used pharmaceutically, they are combined with a “pharmaceutically acceptable carrier” for diagnostic and therapeutic use. These compositions also can be used for the preparation of medicaments for the diagnostic and immunomodulatory methods of the invention.

### Diagnostic and Therapeutic Utilities

The present invention provides diagnostic and immunomodulatory methods using peptides, polynucleotides, antigen-presenting matrices, and host cells (including APCs and educated immune effector cells), i.e., immunomodulatory agents, of the invention.

### Diagnostic Methods

The present invention provides diagnostic methods using antigenic peptide epitopes of the invention. The methods can be used to detect the presence of an antigen-specific CD4⁺ or CD8⁺ T cell which binds an antigenic peptide epitope of the invention.

The diagnostic methods of the invention include: (1) assays to predict the efficacy of an antigenic peptide epitope of the invention; (2) assays to determine the precursor frequency (i.e., the presence and number of) of immune effector cells specific for an antigenic peptide epitope of the invention; and (3) assays to determine the efficacy of an antigenic epitope of the invention once it has been used in an immunomodulatory method of the invention.

Diagnostic methods of the invention are generally carried out under suitable conditions and for a sufficient time to allow specific binding to occur between the antigenic epitope of the invention and an immune effector molecule, such as a TCR, on the surface of an immune effector cell, such as a CD4⁺ or CD8⁺ T cell. “Suitable conditions” and “sufficient time” are generally conditions and times suitable for specific binding. Suitable conditions occur between about 4° C. and about 40° C., preferably between about 4° C. and about 37° C., in a buffered solution, and within a pH range of between 5 and 9. A variety of buffered solutions are known in the art, can be used in the diagnostic methods of this invention, and include, but are not limited to, phosphate-buffered saline. Sufficient time for binding and response will generally be between about 1 second and about 24 hours after exposure of the sample to the antigenic peptide epitope of the invention.

In some embodiments, the invention provides diagnostic ssays to predict the efficacy of an antigenic peptide epitope of the invention. In some of these embodiments, defined T cell epitopes are used to clinically characterize tumors and viral pathogens in order to determine, in advance, the predicted efficacy of an in vivo vaccine trial. This can be achieved by a simple proliferation assay of a patient's peripheral blood mononuclear cells using defined T cell epitopes as stimulators. Peptides which elicit a response are viable vaccine candidates for that patient.

In other embodiments, assays are provided to determine the precursor frequency (i.e., the presence and number of) of resting (naïve) immune effector cells specific for an antigenic peptide epitope of the invention and which therefore have the potential to become activated. In these embodiments, an antigen-presenting cell bearing on its surface an antigenic peptide epitope of the invention is used to detect the presence of immune effector cells in a biological sample which bind specifically to the epitope. A functional assay is used to determine (and quantitate) the antigen-specific immune effector cells.

In other embodiments, the efficacy of an immunomodulatory method, including immunomodulatory methods of the invention, in modulating an immune response to an antigenic epitope of the invention. These diagostic assays are also useful to assess or monitor the efficacy of an immunotherapeutic agent. In some of these embodiments, the method allows detection of immune effector cells, which may be activated CD4⁺ or CD8⁺ T cells, which have become activated or anergized as a result of exposure to an antigenic peptide epitope. A sample containing cells from a subject can be tested for the presence of CD4⁺ or CD8⁺ T cells which have become activated or anergized as a result of binding to a given antigenic peptide epitope of the invention. In some embodiments, the method comprises the steps of: (a) contacting an immobilized antigen-presenting matrix which presents an antigenic peptide epitope of the invention on its surface bound to a Class I or Class II MHC molecule with a biological sample under suitable conditions and for a time sufficient to allow binding of an immune effector cell which bears on its surface an antigen receptor specific for the peptide, thereby immobilizing the antigen-specific immune effector cell; and (b) contacting the immobilized immune effector cell with a detectably labeled molecule, such as an antibody, which specifically binds the immune effector cell. In other embodiments, the method comprises the steps of (a) contacting an immobilized antigen-presenting matrix which presents an antigenic peptide epitope of the invention on its surface bound to a Class I or Class II MHC molecule with a biological sample under suitable conditions and for a time sufficient to allow binding of an immune effector cell which bears on its surface an antigen receptor specific for the peptide, thereby immobilizing the antigen-specific immune effector cell; and (b) performing a functional assay on the immobilized immune effector cell. An immobilized antigen-presenting matrix can be an antigen-presenting matrix immobilized on a solid support including, but not limited to, plates, chips, and beads. Once the immune effector cell is bound to the immobilized antigenic peptide epitope of the invention, it can be labeled on the basis of characteristic cell surface molecules, including, but not limited to, CD4, CD8, and cell surface markers specific for activated T cells. A variety of cell surface markers specific to populations of immune effector cells are known to those skilled in the art and have been described in numerous publications. See, for example, THE LEUKOCYTE ANTIGEN FACTS BOOK, Barclay et al., eds., 1995, Academic Press. Antibodies to these markers are commercially available from, inter alia, Beckman Coulter. The immobilized immune effector cell can also be characterized by presence of mRNA and/or proteins in the cytosol which are characteristic of a given T cell type in a given activated or anergic state. A characteristic mRNA can be detected by any known means, including, but not limited to, a polymerase chain reaction. A detectably labeled antibody to a cell surface marker can be contacted with the immobilized immune effector cell under suitable conditions and for a time sufficient to allow specific binding. If necessary or desired, the labeled cells can be physically removed from unbound label or excess unbound label can be inactivated. The requirements of an antibody specific for a cell surface marker on an immune effector cell are that the antibody bind specifically and that the antibody not interfere with binding between a TCR and the immobilized antigenic peptide epitope.

Labels which may be employed are known to those skilled in the art and include, but are not limited to, traditional labeling materials such as fluorophores, radioactive isotopes, chromophores, and magnetic particles. Enzyme labels include, but are not limited to, luciferase; a green fluorescent protein (GFP), for example, a GFP from Aequorea victoria, or any of a variety of GFP known in the art; â-galactosidase, chloramphenicol acetyl transferase. See, for example, CURRENT PROTOCOLS IN MOLECULAR BIOLOGY (F. M. Ausubel et al., eds., 1987, and periodic updates). Any assay which detects the label, either by directly or indirectly, is suitable for use in the present invention. Assays include colorimetric, fluorimetric, or luminescent assays, radioimmunoassays or other immunological assays.

### Immunomodulatory Methods

The invention provides methods of modulating an immune response in an individual. Immunomodulatory methods of the invention include methods that result in induction or increase, as well as methods that result in suppression or reduction, of an immune response in a subject, and comprise administering to the subject an effective amount of a peptide (or any immunomodulatory agent) of the invention in formulations and/or under conditions that result in the desired effect on an immune response (or lack thereof) to the peptide. Immunomodulatory methods of the invention include vaccine methods, adoptive immunotherapy, and methods to induce T cell unresponsiveness, or anergy.

An “immunomodulatory agent” for use in the methods of the invention is a molecule, a macromolecular complex, or a cell that modulates an immune response and encompasses: an antigenic peptide or epitope of the invention alone or in any of a variety of formulations described herein; a polypeptide comprising an antigenic peptide or epitope of the invention; a polynucleotide encoding a peptide or polypeptide of the invention; an antigenic peptide of the invention bound to a Class I or a Class II MHC molecule on an antigen-presenting matrix, including an APC and a synthetic antigen-presenting matrix (in the presence or absence of co-stimulatory molecule(s)); an antigenic peptide of the invention covalently or non-covalently complexed to another molecule(s) or macromolecular structure; and an educated, antigen-specific immune effector cell which is specific for a peptide of the invention.

Various methods are known to evaluate T cell activation. CTL activation can be detected by any known method, including but not limited to, tritiated thymidine incorporation (indicative of DNA synthesis), and examination of the population for growth or proliferation, e.g., by identification of colonies. Alternatively, the tetrazolium salt MTT (3-(4,5-dimethyl-thazol-2-yl)-2,5-diphenyl tetrazolium bromide) may be added. Mossman (1983) J. Immunol. Methods 65:55-63; Niks and Otto (1990) J. Immunol. Methods 130:140-151. Succinate dehydrogenase, found in mitochondria of viable cells, converts the MTT to formazan blue. Thus, concentrated blue color would indicate metabolically active cells. In yet another embodiment, incorporation of radiolabel, e.g., tritiated thymidine, may be assayed to indicate proliferation of cells. Similarly, protein synthesis may be shown by incorporation of ³⁵S-methionine. In still another embodiment, cytotoxicity and cell killing assays, such as the classical chromium release assay, may be employed to evaluate epitope-specific CTL activation. To detect activation of CD4+ T cells, any of a variety of methods can be used, including, but not limited to, measuring cytokine production; and proliferation, for example, by tritiated thymidine incorporation

Release of ⁵¹Cr from labeled target cells is a standard assay which can be used to assess the number of peptide-specific CTLs in a biological sample. Tumor cells, or APCs of the invention, are radiolabeled as targets with about 200 μCi of Na₂ ⁵¹CrO₄ for 60 minutes at 37° C., followed by washing. T cells and target cells (˜1×10⁴/well) are then combined at various effector-to-target ratios in 96-well, U-bottom plates. The plates are centrifuged at 100×g for 5 minutes to initiate cell contact, and are incubated for 4-16 hours at 37° C. with 5% CO₂. Release of ⁵¹Cr is determined in the supernatant, and compared with targets incubated in the absence of T cells (negative control) or with 0.1% TRITON™ X-100 (positive control). See, e.g., Mishell and Shiigi, eds. SELECTED METHODS IN CELLULAR IMMUNOLOGY (1980) W.H. Freeman and Co.

The formulation of a peptide of the invention will vary, depending on the desired result. In general, peptides presented on an antigen-presenting matrix by a Class I or Class II MHC molecule, together with the appropriate co-stimulatory molecules, will result in induction of an immune response to the peptide. An anergic (or unresponsive) state may be induced in T lymphocytes by presentation of an antigen by an antigen-presenting matrix (which may be an APC) which contains appropriate MHC molecules on its surface, but which lacks the appropriate co-stimulatory molecules. Any of the various formulations described herein can be used.

Polynucleotides of the invention can be administered in a gene delivery vehicle or by inserting into a host cell which in turn recombinantly transcribes, translates and processed the encoded polypeptide. Isolated host cells containing a polynucleotide of the invention in a pharmaceutically acceptable carrier can be combined with appropriate and effective amount of an adjuvant, cytokine or co-stimulatory molecule for an effective vaccine regimen. In some embodiments, the host cell is an APC, such as a dendritic cell. The host cell can be further modified by inserting of a polynucleotide coding for an effective amount of either or both of a cytokine a co-stimulatory molecule.

The methods of this invention can be further modified by co-administering an effective amount of a cytokine or co-stimulatory molecule to the subject.

The agents provided herein as effective for their intended purpose can be administered to subjects having a disease to be treated with an immunomodulatory method of the invention or to individuals susceptible to or at risk of developing such a disease. When the agent is administered to a subject such as a mouse, a rat or a human patient, the agent can be added to a pharmaceutically acceptable carrier and systemically or topically administered to the subject. Therapeutic amounts can be empirically determined and will vary with the pathology or condition being treated, the subject being treated and the efficacy and toxicity of the therapy.

The amount of a peptide or immune effector cell of the invention will vary depending, in part, on its intended effect, and is ultimately at the discretion of the medical or veterinary practitioner. The factors to be considered include the condition being treated, the route of administration, and nature of the formulation, the mammal's body weight, surface area, age, and general condition and the particular peptide to be administered. A suitable effective dose of peptides of the invention generally lies in the range of from about 0.0001 μmol/kg to about 1000 μmol/kg bodyweight. The total dose may be given as a single dose or multiple doses, e.g., two to six times per day. For example, for a 75 kg mammal (e.g., a human) the dose range would be about 2.25 μmol/kg/day and a typical dose could be about 100 μmol of peptide. If discrete multiple doses are indicated treatment might typically be 25 μmol of a peptide of the invention given up to 4 times per day. In an alternative administrative regimen, peptides of the invention may be given on alternate days or even once or twice a week. A suitable effective dose of an immune effector cell of the invention generally lies in the range of from about 10² to about 10⁹ cells per administration. Cells can be administered once, followed by monitoring of the clinical response, such as diminution of disease symptoms or tumor mass. Administration may be repeated on a monthly basis, for example, or as appropriate. Those skilled in the art will appreciate that an appropriate administrative regimen would be at the discretion of the physician or veterinary practitioner.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including nasal, topical (including transdermal, aerosol, buccal and sublingual), parenteral (including subcutaneous, intramuscular, intravenous and intradermal) and pulmonary. It will also be appreciated that the preferred route will vary with the condition and age of the recipient, and the disease or condition being treated.

### Vaccines for Cancer Treatment and Prevention

In one embodiment, immunomodulatory methods of the present invention comprise vaccines for cancer treatment. These vaccines will be both treatments for affected individuals as well as preventive therapy against recurrence (or establishment of the disease in patients which present with a familial genetic predisposition to it). Inoculation of individuals who have never had the cancer is expected to be quite successful as preventive therapy, even though a tumor antigen-specific CTL response has not yet been elicited, because in most cases high affinity peptides seem to be immunogenic suggesting that holes in the functional T cell repertoire, if they exist, may be relatively rare. Sette, et al. (1994) J. Immunol. 153:5586-5592. In mice, vaccination with appropriate epitopes not only eliminates established tumors but also protects against tumor re-establishment after inoculation with otherwise lethal doses of tumor cells. Bystryn, et al. (1993) Supra.

Recent advances in vaccine adjuvants provide effective means of administering peptides so that they impact maximally on the immune system. Del-Giudice (1994) Experientia 50:1061-1066. These peptide vaccines will be of great value in treating metastatic tumors that are generally unresponsive to conventional therapies. Tumors arising from the homozygous deletion of recessive oncogenes are less susceptible to elimination by a humoral (antibody) response and would thus be treated more effectively by eliciting a cellular, CTL response.

### Adoptive Immunotherapy Methods

The expanded populations of antigen-specific immune effector cells and APCs of the present invention find use in adoptive immunotherapy regimes and as vaccines.

Adoptive immunotherapy methods involve, in one aspect, administering to a subject a substantially pure population of educated, antigen-specific immune effector cells made by culturing naïveimmune effector cells with APCs as described above. In some embodiments, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In a further embodiment, APCs or immune effector cells are administered with an effective amount of a stimulatory cytokine, such as IL-2 or a co-stimulatory molecule.

### Experimental Example 1

Melanoma cell lines, differentially susceptible to lysis by a gp100 specific cytotoxic T lymphocyte (CTL) were subjected to SAGE analysis to determine which SAGE tags were shared amongst the cell lines that were susceptible to lysis against those tags that were absent or less abundant in cell lines that were not susceptible to lysis. Table 2, below shows the phenotypes of melanoma cell lines and for SAGE anlysis.

TABLE 2
PHENOTYPE OF MELANOMA CELL LINES
USED FOR SAGE ANALYSIS

| CELL | | | LYSIS BY |
|---|---|---|---|
| LINE | HLA-A2 | gp100 | ANTI-gp100 CTL |
| 1300MEL | + | + | + |
| 624MEL | + | + | + |
| BA1 | + | − | − |
| A375 | + | − | − |

Ten SAGE tags matched the sorting criteria and were found to be represented at a higher level in cell lines identified as 624mel and 1300mel (that are susceptible to lysis) than in cell lines identified to BA1 and A375 (that are not susceptible to lysis). Two different tags corresponding to the differentially spliced forms of the gp100 mRNA were identified but in addition, 8 other tag sequences were found including a tag corresponding to cdc2-related protein kinase. (Table 3). While gp100 has previously been identified as a target for patient derived T cells, it has not been reported that cdc2-related protein kinase can also be a target for patient derived immune effector cells or antibodies.

TABLE 3
COMPARISON OF MELANOMA CELL LINE
SAGE DATA

| <5 | <5 | >10 | >10 | |
|---|---|---|---|---|
| BA1 | A375 | 624 | 1300 | GENE |
| 0 | 0 | 206 | 92 | gp100 melanocyte lineage-specific antigen |
| 0 | 0 | 65 | 18 | gp100 melanocyte lineage-specific antigen |
| 0 | 0 | 60 | 16 | calpain-skeletal muscle protein |
| 1 | 4 | 18 | 25 | Mitchondrial |
| 1 | 4 | 18 | 11 | Biliary glycoprotein |
| 3 | 3 | 47 | 34 | microsomal epoxide hydrolase gene |
| 3 | 4 | 26 | 14 | NM |
| 3 | 4 | 18 | 13 | NM |
| 4 | 4 | 72 | 27 | cdc2-related protein kinase mRNA |
| 4 | 4 | 20 | 11 | ATP synthase subunit c |

NM = no match

### Experimental Example 2

Melanoma and breast cancer cell lines, exhibiting differential immunoreactivity to an anti-HER-2 antibody as judged by FACS analysis were subjected to SAGE analysis to determine which SAGE tags were shared amongst the cell lines that showed a high mean fluorescence signal that were less abundant in cell lines that showed a lower mean fluorescence signal. Four SAGE tags matched the sorting criteria and were found to be represented at a higher level in cell lines 21PT and 21MT (that show a strong fluorescence signal) than in cell lines MDA-468, SK28, BA1, NM455 and 1300 mel (that show a weaker fluorescence signal) (Table 4). One tag corresponding to HER-2 was identified but in addition, 3 other tag sequences were found including a tag corresponding to integrin alpha-3. While HER-2 has previously been identified as a target for patient derived T cells, it has not been reported that integrin alpha-3 can also be a target for patient derived immune effector cells or antibodies. Thus, the gene encoding integrin alpha-3 or the corresponding gene product or peptide fragments thereof can be used to provoke an immune response to target cells that differentially express integrin alpha-3. While integrin alpha-3 was used for this example, any differentially expressed gene or genes (identified by SAGE) and their corresponding proteins or peptide fragments could be used to provoke an anti-target cell immune response.

TABLE 4
IDENTIFICATION OF THE ANTIGEN
RECOGNIZED BY AN ANTIBODY

| Cell Line | Mean Fluorescence |
|---|---|
| 21PT | 35.2 |
| 21 MT | 33.4 |
| MIDA-468 | 3.1 |
| SK28 | 7.4 |
| BA1 | 8.9 |
| NM455 | 11.1 |
| 1300 | 14.7 |

| | |
|---|---|
| >10 | <5 |
| (A & B) | (C through G) |

| A | B | C | D | E | F | G | Gene |
|---|---|---|---|---|---|---|---|
| 66 | 11 | 2 | 0 | 0 | 0 | 1 | No match |
| 21 | 21 | 1 | 1 | 0 | 1 | 3 | AL0096 |
| 11 | 25 | 0 | 0 | 1 | 2 | 2 | HER2 |
| 11 | 15 | 0 | 0 | 4 | 3 | 0 | integrin alpha-3 |

It is to be understood that while the invention has been described in conjunction with the above embodiments, that the foregoing description and the following examples are intended to illustrate and not limit the scope of the invention. For example, any of the above-noted compositions and/or methods can be combined with known therapies or compositions. Other aspects, advantages and modifications within the scope of the invention will be apparent to those skilled in the art to which the invention pertains.

What is claimed is:

1. A method to identify a putative cancer therapeutic comprising the steps of:
(a) identifying a polynucleotide which is uniquely expressed or overexpressed in a target cancer cell as compared with a control non-cancer cell;
(b) determining the protein corresponding to said identified polynucleotide;
(c) determining if said protein, or fragment thereof, is immunogenic,
wherein the ability of said protein to elicit an immune response against said target cancer cell is indicative of a putative cancer therapeutic.

2. The method of claim 1, wherein said immunogenic protein, or fragement thereof, is administered to a subject in a gene delivery vehicle.

3. The method of claim 1, wherein said immunogenic protein, or fragment thereof, is administered to a subject in an antigen presenting cell.

4. The method of claim 1, further comprising the steps of
(a) generating immune effector cells reactive with an immunogenic protein, and
(b) determining if said immune effector cells are immunogenic,
wherein the ability of said immune effector cells to elicit an immune response against said target cancer cell is indicative of a putative cancer therapeutic.

5. The method of claim 1, further comprising the steps of
(a) generating antibodies reactive with an immunogenic protein and,
(b) determining if said antibodies are immunogenic,
wherein the ability of said antibodies to elicit an immune response against said target cancer cell is indicative of a putative cancer therapeutic.

6. The method of claim 5, wherein said antibodies are monoclonal antibodies.

7. A method to design a cancer vaccine from a sample obtained from a subject suffering from cancer, the improvement comprising:
identifying an amino acid sequence which is not previously known to be antigenic, but which is (i) uniquely expressed or overexpressed in a target cancer cell from said subject, as compared with a control non-cancer cell, and
(ii) capable of eliciting an immune response against said target cancer cell.

8. A method for inducing an immune response against a target cell in a subject, comprising delivering to the subject an effective amount of an antigenic peptide that is uniquely expressed or overexpressed in the target cell and has not been previously identified as having the ability to induce an immune response in the subject, whereby an immune response is mounted against the target cell.

9. The method of claim 8, wherein the peptide is delivered as a sequence of amino acids.

10. The method of claim 8, wherein the peptide is delivered as a polynucleotide that encodes the antigenic peptide.

11. The method of claim 8, wherein the uniquely or overexpressed polynucleotide is identified by the method comprising:
(a) obtaining a set of polynucleotides representing gene expression in a target cell;
(b) obtaining a set of polynucleotides representing gene expression in a control cell;
(c) identifying a unique or overexpressed polynucleotide in the target cell as compared to the control cell; and
(d) identifying a unique or overexpressed polynucleotide which is capable of eliciting an immune response in the subject.

12. The method of claim 8, further comprising administering an effective amount of a cytokine and/or co-stimulatory molecule to the subject.

13. The method of claim 10, wherein the polynucleotide is administered to the subject in a gene delivery vehicle.

14. The method of claim 10, wherein the polynucleotide is administered to the subject in a host cell.

15. The method of claim 14, wherein the host cell is a antigen presenting cell.

16. The method of claim 14 or 15, further comprising administering an effective amount of a cytokine and/or co-stimulatory molecule to the subject.

17. A method for enhancing an immune response in a subject against a target cell, comprising administering to the subject an effective amount of an immune effector cell that was raised against an antigenic peptide that is uniquely expressed or overexpressed in the target cell and has not been previously identified as having the ability to induce an immune response in the subject, whereby an immune response is mounted against the target cell.

18. The method of claim 17, wherein the uniquely or overexpressed polynucleotide is identified by the method comprising:
(a) obtaining a set of polynucleotides representing gene expression in a target cell;
(b) obtaining a set of polynucleotides representing gene expression in a control cell;
(c) identifying a unique or overexpressed polynucleotide in the target cell as compared to the control cell; and
(d) identifying a unique or overexpressed polynucleotide which is capable of eliciting an immune response in the subject.

19. The method of claim 17, further comprising administering an effective amount of a cytokine and/or co-stimulatory molecule to the subject.

20. The method of claim 17, wherein said immune effector cell is a cytotoxic T lymphocyte.

21. A method for enhancing an immune response in a subject against a target cell, comprising administering to the subject an effective amount of an antibody that was raised against an antigenic peptide that is uniquely expressed or overexpressed in the target cell and has not been previously identified as having the ability to induce an immune response in the subject, whereby an immune response is mounted against the target cell.

22. The method of claim 21, wherein the uniquely or overexpressed polynucleotide is identified by the method comprising:
(a) obtaining a set of polynucleotides representing gene expression in a target cell;
(b) obtaining a set of polynucleotides representing gene expression in a control cell;
(c) identifying a unique or overexpressed polynucleotide in the target cell as compared to the control cell; and
(d) identifying a unique or overexpressed polynucleotide which is capable of eliciting an immune response in the subject.

23. The method of claim 21, further comprising administering an effective amount of a cytokine and/or co-stimulatory molecule to the subject.

24. The method of claim 21, wherein said antibody is a monoclonal antibody.
